SEQUENCE LISTING (8 sequences)

SEQ ID NO: 1 - DNA, 2229 bp, Danio rerio
  CDS (1)..(2229)
atgatgacgtttctttgcacacggtctggtcgccgtgctagtggtgtggagtgcagaatcgccgctgaacgcgcttactttcgagtgcgtggactcccggttgccaatatgattggctggtggccacgcctctgcccagtgatgtcactggcactgctgtgggcgtgttcagtgggggcgggttcagactgcaaatctgtggccattgagactgacagccgcatagcagaacaaacacagcagcgtcacctacaggctctgttcgacaagtatggccagaacggcagcatctccctagaaggcctcttcaacctacttaaaggggtcgggcttgaccgcatccggaaagtgatggtgcatcatcctggaaatgcccataatcacacacacacgcatgatcacacacacactcatgtggacaaactcacggcgcacacacatccggtcaccaccaagaagggagacatggatcacagcgtggagaagagtgaccctgtcccaaaagcacagccagatcctgcctctgggaagaaaagccagtcagatgcgcatcacaacctgtacatgaagatgaaccaggaatccaccacagctttgactacgccgtcatatgttaccagatcacggcggaccaatcgcagcgccgattatgattttacacaggaccacgcctcctttagccccagtcagcccaatgtgacacactcaaaccacacccatcatgatgaggacacgcccacacaccagcatgatgaccatgatgagcacgaacatgcccgtgctagtttagggtgtcaaaatgcctccacgatcctgcagacgcatggcatgagaaaggaagcaagtctctcagttaaggacttcagtttcctctgccctgctcttctcatgcagattgattccaagtcttgcatcgtgcatgaagacgaggacgagcattcagatcattcccatcatcacaaacaccaccaccatcatcatgatcaccaacacctgcagcatccacataaccacaccaatggaagaggccagaggaacactccagtctacatcgcatggcttggagggtttctctccatcactctgatcagtttgctggcgttggttggtgtggttttgatcccactcatgaacagagtttgcttcaacttcctgctgagcttcctggtggcccttgcagtgggcactctgagcggagacgctctcctccacctcataccacattctcagggtcatcaccatcacggccactctgaagagcacgctgaagaggaggactcccttcgccctgtgtggaccggactcacagctctaagtggagtttacatcatgttcctcatcgaacacttcctgacccttggcaaaatgtacaaagacaaaaaccagaaggtgcagaagagggttgatctcaccacagaagttttggagtctgagaaactgccatcattagaagaaaatgatgtcaaaattgaagctgctgaaacgaatggtgggcgtgcactggcagaggaggaggaggtgatgttgggggcggagctctacaacgacatagactgcgagaacaaatgccactcccacttccatgacacggtcggccaatcggatgagcagcatcatcatcatcacgactaccaccacatactgcatcatcaccactcccagaaccaccacccgcacacacacacgcacagacacacacactcctactcgcagcagcactttgagcaggctggtgtggccacactcgcctggatggtcatcatgggagacggactgcacaacttcagtgatggacttgccataggggcggctttcacagaaggtttgtccagtggtcttagtacctcagtcgctgtgttctgccatgagcttcctcatgaactcggtgattttgccgtcctactgaaagccggtatgtcagttcgacaggccatgctgtataatctgctgtcagcactgatgggatatctgggcatgatcatcgggattctcatcggacattatgctgaaaatgttgccacatggatctttgctctcacagctgggttattcatgtacgtcgcgctcgtggacatggtacctgagatgctgcacaatgacgcgagcgaagcaggtttcagtcactacggcttcttcctcctgcagaacgctgggatactcctaggcttcggcatcatgcttatcattgctgtctttgaggacaggatccaactggacttaggttactga

SEQ ID NO: 2 - protein, 742 aa, Danio rerio
MMTFLCTRSGRRASGVECRIAAERAYFRVRGLPVANMIGWWPRLCPVMSLALLWACSVGAGSDCKSVAIETDSRIAEQTQQRHLQALFDKYGQNGSISLEGLFNLLKGVGLDRIRKVMVHHPGNAHNHTHTHDHTHTHVDKLTAHTHPVTTKKGDMDHSVEKSDPVPKAQPDPASGKKSQSDAHHNLYMKMNQESTTALTTPSYVTRSRRTNRSADYDFTQDHASFSPSQPNVTHSNHTHHDEDTPTHQHDDHDEHEHARASLGCQNASTILQTHGMRKEASLSVKDFSFLCPALLMQIDSKSCIVHEDEDEHSDHSHHHKHHHHHHDHQHLQHPHNHTNGRGQRNTPVYIAWLGGFLSITLISLLALVGVVLIPLMNRVCFNFLLSFLVALAVGTLSGDALLHLIPHSQGHHHHGHSEEHAEEEDSLRPVWTGLTALSGVYIMFLIEHFLTLGKMYKDKNQKVQKRVDLTTEVLESEKLPSLEENDVKIEAAETNGGRALAEEEEVMLGAELYNDIDCENKCHSHFHDTVGQSDEQHHHHHDYHHILHHHHSQNHHPHTHTHRHTHSYSQQHFEQAGVATLAWMVIMGDGLHNFSDGLAIGAAFTEGLSSGLSTSVAVFCHELPHELGDFAVLLKAGMSVRQAMLYNLLSALMGYLGMIIGILIGHYAENVATWIFALTAGLFMYVALVDMVPEMLHNDASEAGFSHYGFFLLQNAGILLGFGIMLIIAVFEDRIQLDLGY

SEQ ID NO: 3 - DNA, 2268 bp, Homo sapiens
  CDS (1)..(2268)
atggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccatgatcatcactctcaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttcaactatctctgtccagccatcatcaaccaaattgatgctagatcttgtctgattcatacaagtgaaaagaaggctgaaatccctccaaagacctattcattacaaatagcctgggttggtggttttatagccatttccatcatcagtttcctgtctctgctgggggttatcttagtgcctctcatgaatcgggtgtttttcaaatttctcctgagtttccttgtggcactggccgttgggactttgagtggtgatgcttttttacaccttcttccacattctcatgcaagtcaccaccatagtcatagccatgaagaaccagcaatggaaatgaaaagaggaccacttttcagtcatctgtcttctcaaaacatagaagaaagtgcctattttgattccacgtggaagggtctaacagctctaggaggcctgtatttcatgtttcttgttgaacatgtcctcacattgatcaaacaatttaaagataagaagaaaaagaatcagaagaaacctgaaaatgatgatgatgtggagattaagaagcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaagtctacaatgaatatgtacccagagggtgcaagaataaatgccattcacatttccacgatacactcggccagtcagacgatctcattcaccaccatcatgactaccatcatattctccatcatcaccaccaccaaaaccaccatcctcacagtcacagccagcgctactctcgggaggagctgaaagatgccggcgtcgccactttggcctggatggtgataatgggtgatggcctgcacaatttcagcgatggcctagcaattggtgctgcttttactgaaggcttatcaagtggtttaagtacttctgttgctgtgttctgtcatgagttgcctcatgaattaggtgactttgctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctag

SEQ ID NO: 4 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSDHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 5 - DNA, 2298 bp, Mus musculus
  CDS (1)..(2298)
atggccacagatttatctgtaatcatgatcttgacctttgccctttgggttacaagcccccttcatgaactacaatcaacagctgctttctctcagactactgagaaaataaattcaaattgggaacctggtgttaatgttgacttggcagttaccatgcagcgacaccatctgcagcagctattctaccgctacggagagaatgattccttgtctgttgaaggcttcagaaaattgcttcagaacataggcatagataagattaaaagagtccatatacaccatgaccacgagcatcatgctgaccacgagcatcactcggaccatgagcatcactcggaccacgagcatcactcggaccacgagcatcactcggaccacgagcatcactcggaccacgagcaccattcccaccgcagtcacacggttgctggtaaaaacaatcggaaagccttttgtccagaccttgactctgataattcaggtaaaaatcctagaactagtctagggaaaggatctcgcccagcagagcacatgaatggtaggaggaacatcaaggagagtgcaagctctagtgaagtgacctcggcggtatacaacgctgtctctgaaggaactcgctttgtagagacaatagagactccaaaacctgggagacgcaccaaagatgtaaacccttctaccccacccagcatcacggagaaaagccgagtgggccggctgagtcggctagctaggaagaaaagcaatgagtctgtgagtgagcccagaaagagctttatgtattccagaaacacaaatgacaatattcaggagtgtttcaatacaaccaagctgctgacatcccatggcatgagcatccaggctctgttgaatgcaacggaatttaactatctctgcccagccatcatcaatcaaattgatgctcgggcttgtctgattcatacagcaagtgagaagaaggcagaaatccctccaaagacctattctttacaaatagcctggcttggtggcttcatagccatttccatcatcagtttcctgtctctgctgggagtcatcttggtgccactcatgaaccgggtatttttcaagttcctgctgagcttcctcgtggcgctggccgtcggaacgctgagtggcgatgctctgttacatcttctcccacactctcatgcaagtcatcagcacagtcatagccatgaagagccagcgatggaaatgaaaagaggccccctgttcagccacctgtcggctcagaatatagaagaaagctcctattttgattccacgtggaaaggtctgacggctctagggggcttatatttcatgtttcttgtggaacacgtactcacactgatcaagcaatttaaagataagaaaaagaagaatcaaaagaaacctgaaaatgatgaggatgtggagagcaagaagcagctgtccaaatacgactctcagctttcctcaaatgaagagaaggtggacccaggggaacgacctgaaagctatctgcgagccgactcccaagagccctccccctttgattcccagcagccgacgatgttggaagaggaagaggtcatgatagcccatgcacacccacaagaagtctacaatgaatatgtgcccaggggctgcaagaacaagtgccattcacacttccacgatacgctgggccagtccgacgacctcatccaccaccatcacgactaccatcacattctgcaccaccaccaccaccagaaccaccaccctcacagccacagccagcgctactctcgagaggagctgaaggacgccggcattgccacattggcctggatggtgatcatgggcgacgggctgcacaatttcagtgacggccttgctattggtgctgccttcaccgagggtttgtccagtgggctaagcacctctgtcgctgtgttctgtcatgaactgcctcatgaactaggtgactttgctgttttgctaaaggctggcatgactgtcaagcaggctgtgctctataatgctctgtcagccatgttggcctaccttggaatggcaacagggatattcatcgggcattatgcagaaaatgtttctatgtggatattcgcactcactgccggcttgttcatgtatgtcgctctggttgacatggtgcctgagatgttgcacaatgatgctagtgatcacggatgcagccgttggggatatttcttcctgcagaatgctgggatacttctcggttttggaattatgttactcatttccatatttgagcataaaattgtgtttcgtataaatttctaa

SEQ ID NO: 6 - protein, 765 aa, Mus musculus
MATDLSVIMILTFALWVTSPLHELQSTAAFSQTTEKINSNWEPGVNVDLAVTMQRHHLQQLFYRYGENDSLSVEGFRKLLQNIGIDKIKRVHIHHDHEHHADHEHHSDHEHHSDHEHHSDHEHHSDHEHHSDHEHHSHRSHTVAGKNNRKAFCPDLDSDNSGKNPRTSLGKGSRPAEHMNGRRNIKESASSSEVTSAVYNAVSEGTRFVETIETPKPGRRTKDVNPSTPPSITEKSRVGRLSRLARKKSNESVSEPRKSFMYSRNTNDNIQECFNTTKLLTSHGMSIQALLNATEFNYLCPAIINQIDARACLIHTASEKKAEIPPKTYSLQIAWLGGFIAISIISFLSLLGVILVPLMNRVFFKFLLSFLVALAVGTLSGDALLHLLPHSHASHQHSHSHEEPAMEMKRGPLFSHLSAQNIEESSYFDSTWKGLTALGGLYFMFLVEHVLTLIKQFKDKKKKNQKKPENDEDVESKKQLSKYDSQLSSNEEKVDPGERPESYLRADSQEPSPFDSQQPTMLEEEEVMIAHAHPQEVYNEYVPRGCKNKCHSHFHDTLGQSDDLIHHHHDYHHILHHHHHQNHHPHSHSQRYSREELKDAGIATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGILLGFGIMLLISIFEHKIVFRINF

SEQ ID NO: 7 - DNA, 25 bp, Artificial
  antisense
cggaaacagcgcgagtgtcttttgt

SEQ ID NO: 8 - DNA, 25 bp, Artificial
  antisense
accgtgtgcaaagaaacgtcatcat

(19) United States
(12) Patent Application Publication
(10) Pub. No.: US 2009/0054316 A1
(43) Pub. Date: Feb. 26, 2009

(54) AGENT FOR CONTROL OF FUNCTION OF ANTIGEN-PRESENTING CELL

(75) Inventors: Toshio Hirano, Kanagawa (JP); Hidemitsu Kitamura, Kanagawa (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi, Saitama (JP)

(21) Appl. No.: 11/815,071

(22) PCT Filed: Jan. 31, 2006
(86) PCT No.: PCT/JP2006/301933

§ 371 Date: Aug. 29, 2007

(30) Foreign Application Priority Data
Jan. 31, 2005 (JP) 2005-023200

(51) Int. Cl. A61K 38/17; C07K 14/47; C12N 5/08

(52) U.S. Cl. 514/12; 530/350; 435/375

(57) ABSTRACT

The present invention provides an agent capable of controlling the function of antigen-presenting cell. The present invention provides an agent for controlling the function of antigen-presenting cell, comprising a substance capable of controlling an intracellular zinc ion concentration, particularly, a zinc ion, a zinc ion chelator, a substance for regulating the expression and/or function of a zinc ion-requiring protein, or a substance for regulating the expression and/or function of a zinc ion transporter, as an active ingredient.

### TECHNICAL FIELD

The present invention relates to an agent capable of controlling the function of an antigen-presenting cell. More specifically, the present invention relates to an agent capable of controlling an immune response reaction mediated by activation of an antigen-presenting cell.

### BACKGROUND ART

One of the systems for maintaining human health is the immune system. Controlling immune responses is of paramount importance from the viewpoint of development of a vaccine aiming at protection against infections, treatment of autoimmune diseases, conquest of cancer and the like, and suppression of graft rejection in organ transplant. To date, for the purpose of controlling the immune responses, various immunopotentiators, for example, microbial components such as BCG, have been used and immunosuppressants derived from microbial components and synthetic steroids have been administered to the human body. However, these microbial components and synthetic steroids involve various adverse reactions, and are potentially harmful to the human body. Also, some problems with the use thereof as drugs, including attenuation of the effects thereof during long-term use, have been pointed out to date.

Antigen-presenting cells such as dendritic cells have a central role in the control of the intensity and quality of immune responses, and are applied to immunotherapies for autoimmune diseases, allergic diseases, and cancer, and the like. Research is also ongoing into culture methods for controlling the growth or activation of antigen-presenting cells, and the like, but still there is a demand for a method of more efficiently and specifically controlling the function of an antigen-presenting cell.

The prior art is described in detail in Guermonprez P, Valladeau J, Zitvogel L, Thery C, Amigorena S. Antigen presentation and T cell stimulation by dendritic cells. Annu Rev. Immunol. 20, 621-627 (2002), Banchereau J, Briere F, Caux C, Davoust J, Lebecque S, Liu Y J, Pulendran B, Palucka K. Immunobiology of dendritic cells. Annu Rev. Immunol. 18, 761-781 (2000), and Steinman R M, Mellman I. Immunotherapy: bewitched, bothered, and bewildered no more. Science 305, 197-200 (2004).

### DISCLOSURE OF THE INVENTION

It is an object of the present invention to provide a novel agent for controlling the function of an antigen-presenting cell, specifically to provide an agent capable of controlling immune responses mediated by activation of an antigen-presenting cell (controlling as mentioned herein includes both enhancement and suppression depending on the object). It is another object of the present invention to apply the agent to various diseases, including treatment of autoimmune diseases, treatment of allergic diseases, suppression of graft rejection in organ transplant, and the like, and still other objects are to develop a method of enhancing immune responses to cancer, and to efficiently develop a vaccine.

The present inventors diligently investigated in view of the above-described objects, and found that by controlling the zinc ion level, the function of an antigen-presenting cell such as a dendritic cell can be controlled. Furthermore, the present inventors confirmed that the function control of an antigen-presenting cell by controlling the zinc ion concentration was reversible, and developed the present invention. Accordingly, the present invention provides:

[1] An agent for controlling the function of an antigen-presenting cell, comprising a substance capable of controlling the intracellular zinc ion concentration as the active ingredient.
[2] The agent described in [1] above, wherein the substance capable of controlling the zinc ion concentration is a zinc ion.
[3] The agent described in [2] above, wherein the zinc ion is introduced into a cell through a zinc ionophore.
[4] The agent described in [1] above, wherein the substance capable of controlling the zinc ion concentration is a zinc ion chelator.
[5] The agent described in [4] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA) and N-(6-methoxy-8-quinolyl)-p-toluenesulfonamide (TSQ).
[6] The agent described in [1] above, wherein the substance capable of controlling the zinc ion concentration is a substance regulating the expression and/or function of a zinc ion-requiring protein.
[7] The agent described in [6] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[8] The agent described in [1] above, wherein the substance capable of controlling the zinc ion concentration is a substance regulating the expression and/or function of a zinc ion transporter.
[9] The agent described in [8] above, wherein the zinc ion transporter is at least one selected from the group consisting of human ZIPs including LIV family and human CDFs.
[10] The agent described in any of [1] to [9] above, wherein the antigen-presenting cell is at least one selected from the group consisting of a dendritic cell, a macrophage, a Langerhans' cell, a B cell, a thymic epithelial cell, a synovial cell, a vascular endothelial cell and a keratinocyte.
[11] The agent described in [10] above, wherein the antigen-presenting cell is a dendritic cell.
[12] A prophylactic and/or therapeutic drug for a disease mediated by the immune system, comprising the function controlling agent described in any of [1] to [11] above.
[13] The prophylactic and/or therapeutic drug described in [12] above, wherein the disease mediated by the immune system is at least one selected from the group consisting of autoimmune disease, allergic disease, graft rejection in organ transplant, an immune response reduction due to infection or tumorigenesis, and an abnormal immune response during vaccination.
[14] A prophylactic and/or therapeutic drug for a disease mediated by the immune system, comprising a substance capable of controlling the intracellular zinc ion concentration as the active ingredient.
[15] The prophylactic and/or therapeutic drug described in [14] above, wherein the substance capable of controlling the zinc ion concentration is a zinc ion.
[16] The prophylactic and/or therapeutic drug described in [15] above, wherein the zinc ion is introduced into a cell through a zinc ionophore.
[17] The prophylactic and/or therapeutic drug described in [14] above, wherein the substance capable of controlling the zinc ion concentration is a zinc ion chelator.
[18] The prophylactic and/or therapeutic drug described in [17] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA) and N-(6-methoxy-8-quinolyl)-p-toluenesulfonamide (TSQ).
[19] The prophylactic and/or therapeutic drug described in [14] above, wherein the substance capable of controlling the zinc ion concentration is a substance regulating the expression and/or function of a zinc ion-requiring protein.
[20] The prophylactic and/or therapeutic drug described in [19] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[21] The prophylactic and/or therapeutic drug described in [14] above, wherein the substance capable of controlling the zinc ion concentration is a substance regulating the expression and/or function of a zinc ion transporter.
[22] The prophylactic and/or therapeutic drug described in [21] above, wherein the zinc ion transporter is at least one selected from the group consisting of human ZIPs including LIV family and human CDFs.
[23] The prophylactic and/or therapeutic drug described in any of [14] to [22] above, wherein the disease mediated by the immune system is at least one selected from the group consisting of autoimmune disease, allergic disease, graft rejection in organ transplant, an immune response reduction due to infection or tumorigenesis and an abnormal immune response during vaccination.
[24] The prophylactic and/or therapeutic drug described in any of [14] to [23] above, wherein the antigen-presenting cell is at least one selected from the group consisting of a dendritic cell, a macrophage, a Langerhans' cell, a B cell, a thymic epithelial cell, a synovial cell, a vascular endothelial cell and a keratinocyte.
[25] The prophylactic and/or therapeutic drug described in [24] above, wherein the antigen-presenting cell is a dendritic cell.
[26] A method of controlling the function of an antigen-presenting cell, comprising controlling the intracellular zinc ion concentration in vitro.
[27] The method described in [26] above, wherein the zinc ion concentration is controlled by introducing a zinc ion into a cell.
[28] The method described in [27] above, wherein the zinc ion is introduced into the cell through a zinc ionophore.
[29] The method described in [26] above, wherein the zinc ion concentration is controlled by a zinc ion chelator.
[30] The method described in [29] above, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA) and N-(6-methoxy-8-quinolyl)-p-toluenesulfonamide (TSQ).
[31] The method described in [26] above, wherein the zinc ion concentration is controlled by regulating the expression and/or function of a zinc ion-requiring protein.
[32] The method described in [31] above, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.
[33] The method described in [26] above, wherein the zinc ion concentration is controlled by regulating the expression and/or function of a zinc ion transporter.
[34] The method described in [33] above, wherein the zinc ion transporter is at least one selected from the group consisting of human ZIPs including LIV family and human CDFs.
[35] The method described in any of [26] to [34] above, wherein the antigen-presenting cell is at least one selected from the group consisting of a dendritic cell, a macrophage, a Langerhans' cell, a B cell, a thymic epithelial cell, a synovial cell, a vascular endothelial cell and a keratinocyte.
[36] A screening method for a compound capable of controlling the function of an antigen-presenting cell, comprising measuring the intracellular zinc ion concentration.
[37] The method described in [36] above, wherein the antigen-presenting cell is at least one selected from the group consisting of a dendritic cell, a macrophage, a Langerhans' cell, a B cell, a thymic epithelial cell, a synovial cell, a vascular endothelial cell and a keratinocyte.
[38] The method described in [37] above, wherein the antigen-presenting cell is a dendritic cell.
[39] A use of a substance capable of controlling the intracellular zinc ion concentration, for producing a function controlling agent for an antigen-presenting cell.
[40] A use of the function controlling agent described in any of [1] to [11] above, for producing a prophylactic and/or therapeutic drug for a disease mediated by the immune system.
[41] A use of a substance capable of controlling the intracellular zinc ion concentration, for producing a prophylactic and/or therapeutic drug for a disease mediated by the immune system.

### BRIEF DESCRIPTION OF THE DRAWINGS

FIG. 1 is a drawing showing that when dendritic cells are treated with a zinc ion chelator, the expression of MHC class I, class II, and CD86 molecules on the cell surface thereof is enhanced.

FIG. 2 is a drawing showing that when dendritic cells are treated with a zinc ion and a zinc ionophore, LPS-stimulated enhancement of expression of MHC II and CD86 molecules and IL-12 production in the dendritic cells are suppressed.

FIG. 3 is a drawing showing that on dendritic cells allowed to express mLiv1 using a retrovirus vector, LPS-stimulated enhancement of expression of MHC class II molecule is suppressed.

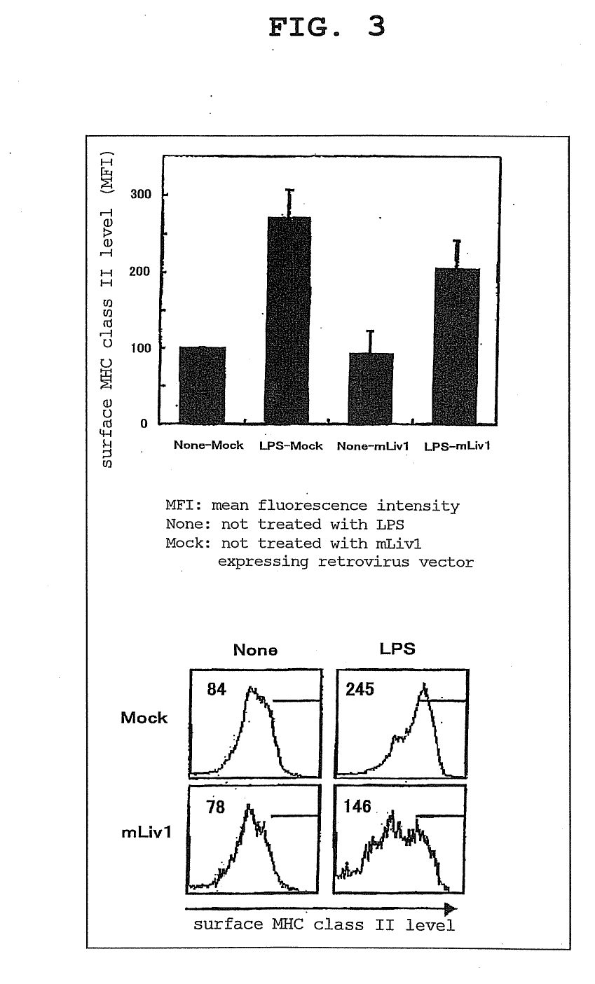

### BEST MODE FOR EMBODYING THE INVENTION

In the present invention, by “controlling a zinc ion concentration” is not only literally meant making the intracellular zinc ion concentration (amount) increase or decrease, but also meant an effect which can ultimately induce the same phenomena as those caused by an increase or decrease of the intracellular zinc ion concentration, and in such a case, the wording is not limited by the level of the intracellular zinc ion concentration. In the present invention, a cell to be a target of control of the zinc ion concentration is an antigen-presenting cell such as a dendritic cell, a macrophage, a Langerhans' cell, a B cell, or a thymic epithelial cell, or a cell expected to acquire antigen-presenting capability, such as a synovial cell, a vascular endothelial cell, or a keratinocyte. The cell is preferably a dendritic cell. Herein, a cell to be a target of control is sometimes referred to as “an antigen-presenting cell” for the sake of convenience.

A “substance capable of controlling an intracellular zinc ion concentration” includes a “zinc ion”, a “zinc ion chelator”, a “zinc ionophore”, a “substance regulating the expression and/or function of a zinc ion-requiring protein”, a “substance regulating the expression and/or function of a zinc ion transporter” and the like. The “zinc ion” and “zinc ionophore” can directly increase the intracellular zinc ion concentration of the antigen-presenting cell by addition thereof, whereas the “zinc ion chelator” is capable of directly reducing the zinc ion concentration in an antigen-presenting cell by being added, whereby the function of an antigen-presenting cell can be controlled. The “substance regulating the expression and/or function of a zinc ion-requiring protein” and “a substance regulating the expression and/or function of a zinc ion transporter” are capable of altering the action of the zinc ion on an antigen-presenting cell and controlling the function of the antigen-presenting cell by regulating the expression and/or function of the zinc ion-requiring protein, or by regulating the expression and/or function of the zinc ion transporter. When the “substance regulating the expression and/or function of a zinc ion-requiring protein” or the “substance regulating the expression and/or function of a zinc ion transporter” is a substance regulating in the direction of promoting the expression or function, the responsiveness of the cell to zinc ions can be more enhanced, and when the substance is a substance regulating in the direction of suppressing the expression or function, the responsiveness of the cell to zinc ions can be suppressed more. In the present invention, “controlling” the zinc ion concentration means both positive and negative control.

An antigen-presenting cell presents 1) an exogenous substance (foreign substance) derived from infection and the like and 2) an endogenous protein resulting from tumorigenesis of one's own body or a cell, viral infection and the like to a cell having TCR (for example, CD4⁺T cell, CD8⁺T cell, DNT cell, NKT cell and the like). Therein, protection against infections occurs and cells that are undesirable for one's own body are removed, on the basis of distinguishing between self and non-self. In the present invention, by “controlling” these functions of an antigen-presenting cell, an immune response preferable for the maintenance of life is induced. “Controlling” the function of an antigen-presenting cell more specifically means defining the quality and quantity of an immune response after antigen presentation. As used herein, “quality” means control of responses of Th1 and Th2 cells, immunotolerance and the like in CD4⁺T cells, or control of responses of CTL, which has antigen-specific cytotoxic activity, immunotolerance and the like in CD8⁺T cells; “quantity” literally means the intensity of the immune response. Also, “controlling” the function of an antigen-presenting cell means the activation or inactivation of the antigen-presenting cell per se. Specifically, “controlling” the function of an antigen-presenting cell means function controlling of one or more items selected from among 1) control of expression of MHC class I or II molecule, 2) control of expression of a co-stimulatory molecule such as CD80, CD86, or CD40, 3) control of expression of a cytokine such as IL-1, IL-6, IL-10, IL-12, IFN-α/β, TNF-α, or TGF-β, 4) control of uptake of an exogenous or endogenous antigen, 5) control of migration capability of an antigen-presenting cell, and the like.

The “zinc ion” is introduced into the antigen-presenting cell through a zinc ionophore. Namely, the zinc ion is introduced in the form of a complex with the zinc ionophore into the antigen-presenting cell. The zinc ionophore includes a variety of compounds commonly used in the art, preferably those commercially available. It is exemplified by zinc pyrithione, heterocyclic amine, dithiocarbamate, vitamins and the like.

The “zinc ion chelator” is a substance that can form a complex with the zinc ion in the antigen-presenting cell, thereby removing the zinc ion from the cell, and includes, for example, 2,3-dimercapto-1-propanesulfonic acid (DMPS), N,N,N′,N′-tetrakis(2-pyridylmethyl)ethylenediamine (TPEN), ethylenediamine (EDTA), N-(6-methoxy-8-quinolyl)-p-toluenesulfonamide (TSQ) and the like. All of them are commercially available. The amount of the zinc ion chelator as a substance capable of controlling the concentration of the zinc ion to be comprised as an active ingredient in the function controlling agent of the present invention for an antigen-presenting cell or the prophylactic and/or therapeutic drug of the present invention for a disease mediated by the immune system (hereinafter, sometimes simply referred to as “prophylactic and/or therapeutic drug”) is appropriately determined within a range in which the control of the intracellular zinc ion concentration is possible, in each agent or drug. One or more types of zinc ion chelators may be comprised therein. When two or more types of zinc ion chelators are comprised, their amounts are appropriately set depending on their types.

The “substance regulating the expression and/or function of a zinc ion-requiring protein” is not particularly limited by its mechanism of action, as long as it can eventually regulate the expression and/or function of a zinc ion-requiring protein, and the regulation may be at the gene level or protein level. The regulation at the gene level includes transcriptional regulation, gene expression regulation and the like. The regulation at the protein level includes metabolic regulation, phosphorylation, dephosphorylation, glycosylation, lipidation, coordinate bond with zinc, degradation, ubiquitination, acetylation and the like. The “zinc ion-requiring protein” is a protein that requires zinc for its functional expression in the antigen-presenting cell, and includes, for example, a protein having a zinc finger, a protein having a RING finger, a protein having an LIM domain, a protein having a PHD zinc finger and the like. The zinc finger is a motif having a unique nucleic acid-binding ability, which can form a higher-order structure only by coordination of amino acids such as cysteine and histidine to the zinc. For example, a protein having a zinc finger is exemplified by a transcription factor Snail which is a master regulator in a phenomenon called epithelial-mesenchymal transition (phenomenon in which cells usually tightly adhering to each other acquire mobility by releasing the adhesion to their adjacent cells and migrate to other sites during morphogenesis in the early development of humans and other organisms and during wound healing and cancer metastasis and the like), PKCα, GEF-H1, HDAC, SQSTM1 and the like. The RING finger may be said to be a variation of the zinc finger and it has been suggested that it is involved in a protein-protein interaction. The reported protein having the RING finger includes those having ubiquitin-protein ligase E3 activity, those exhibiting ubiquitin-conjugating enzyme E2 binding, and those exhibiting TRAF6 (a signaling molecule of TNFα) binding. The LIM domain consists of 60 amino acids with the positions of 6 cysteines and 1 histidine being conserved. Although this domain is structurally very similar to the zinc finger, its DNA binding ability has not been known. It also functions as a domain mediating the binding to PKC. A protein having the LIM domain is exemplified by Paxillin, Zyxin and the like, which are involved in cytoskeletal control. The PHD zinc finger is a zinc finger-like domain in nuclear proteins suggested to be involved in chromatin-mediated transcriptional control and is expected to have the DNA binding ability. A protein having a PHD zinc finger is exemplified by Jade-1 which is involved in histone acetylation and the like.

The “substance regulating the expression and/or function of a zinc ion-requiring protein” may be a known compound or a novel compound to be developed in the future. It may be a low-molecular-weight compound or a high-molecular-weight compound. In this context, the low-molecular-weight compound is a compound having a molecular weight of less than approximately 3000, which includes, for example, an organic compound and a derivative thereof and an inorganic compound usually available as a pharmaceutical agent, and refers to a compound and a derivative thereof produced by making full use of organic synthesis or the like, a naturally occurring compound and a derivative thereof, small nucleic acid molecules such as promoter, a variety of metals and the like, and desirably an organic compound and a derivative thereof and a nucleic acid molecule available as a pharmaceutical agent. The high-molecular-weight compound is a compound having a molecular weight of not lower than approximately 3000, which includes, for example, protein, polynucleic acid, polysaccharide and combinations thereof, and is desirably a protein. These low-molecular-weight or high-molecular-weight compounds, if they are known, are commercially available or can be obtained by way of steps such as collection, production and purification according to the respective reported documents. These compounds may be naturally occurring or prepared by genetic engineering, or can also be obtained by semi-synthesis and the like. Specifically, the expression of a zinc ion-requiring protein Snail is regulated by MAPK through TGF-β or FGF (Peinado, H., Quintanilla, M. & Cano, A. Transforming growth factor beta-1 induces snail transcription factor in epithelial cell lines: mechanisms for epithelial mesenchymal transitions. J. Biol. Chem. 278, 21113-23 (2003)). Moreover, Snail is activated by LIV1 (as described below), a zinc ion transporter (Yamashita, S., Miyagi, C., Fukada, T., Kagara, N., Che, Y.-S. & Hirano, T. Zinc transporter LIVI controls epithelial-mesenchymal transition in zebrafish gastrula organizer. Nature 429, 298-302 (2004)). Details for Raf1, GEF-H1, PKCα, phosphatase and metallothionein exemplified as other zinc ion-requiring proteins are respectively described in Jirakulaporn T, Muslin A J. Cation diffusion facilitator proteins modulate Raf-1 activity. J. Biol. Chem. 279, 27807-15 (2004), Krendel M, Zenke F T, Bokoch G M. Nucleotide exchange factor GEF-H1 mediates cross-talk between microtubules and the actin cytoskeleton. Nature Cell Biology 4, 294-301 (2002), Korichneva I, Hoyos B, Chua R, Levi E, Hammerling U. Zinc release from protein kinase C as the common event during activation by lipid second messenger or reactive oxygen. J. Biol. Chem. 277, 44327-31 (2002), Haase H, Maret W. Intracellular zinc fluctuations modulate protein tyrosine phosphatase activity in insulin/insulin-like growth factor-1 signaling. Exp. Cell Research 291, 289-298 (2003), and Seikagaku Jiten (3^{rd} ed.), Tokyo Kagaku Dozin Co., Ltd., pp. 1393 (1998). Details for TRAF6, Paxillin, Zyxin and Jade-1 are respectively described in Kobayashi N, Kadono Y, Naito A, Matsumoto K, Yamamoto T, Tanaka S, Inoue J. Segregation of TRAF6-mediated signaling pathways clarifies its role in osteoclastogenesis. The EMBO J. 20, 1271-1280 (2001), Brown M C, Perrotta J A, Turner C E. Identification of LIM3 as the principal determinant of paxillin focal adhesion localization and characterization of a novel motif on paxillin directing vinculin and focal adhesion kinase binding. J. Cell Biol. 135, 1109-1123 (1996), Sadler I, Crawford A W, Michelsen J W, Beckerle M C. Zyxin and cCRP: two interactive LIM domain proteins associated with the cytoskeleton. J. Cell Biol. 119, 1573-1587 (1992), and Panchenko M V, Zhou M I, Cohen H T. von Hippel-Lindau partner Jade-1 is a transcriptional co-activator associated with histone acetyltransferase activity. J. Biol. Chem. 279, 56032-56041 (2004). Furthermore, details for SQSTM1 and HDAC are respectively described in Joung I, Strominger J L, Shin J. Molecular cloning of a phosphotyrosine-independent ligand of the p56lck SH2 domain. Proc Natl Acad Sci USA, 93, 5991-5995 (1996) and Kawaguchi Y, Kovacs J J, McLaurin A, Vance J M, Ito A, Yao T P. The deacetylase HDAC6 regulates aggresome formation and cell viability in response to misfolded protein stress. Cell 115, 727-738 (2003).

The “substance regulating the expression and/or function of a zinc ion transporter” is not particularly limited by its mechanism of action, as long as it can eventually regulate the expression and/or function of a zinc ion transporter, and the regulation may be at the gene level or protein level. The regulation at the gene level includes transcriptional regulation, gene expression regulation and the like. The regulation at the protein level includes metabolic regulation, phosphorylation, dephosphorylation, glycosylation, lipidation, coordinate bond with zinc, degradation, ubiquitination, acetylation and the like. The “zinc ion transporter” includes, for example, human ZIPs including LIV family (e.g., BAB70848, hZip4, BIGM103, KIAA0062, KIAA1265, hLiv-1, AAH08853, hKE4, XP_{—}208649, hZIP1, hZIP2, hZIP3, BAA92100, BAC04504), human CDFs (e.g., hZnt-5, hZnt-7, hZnt-1, hZnt-6, hZnt-3, hZnt-2, hZnt-8, hZnt-4, hZnt-9) and the like. LIV1 was originally identified as an estrogen-controlled breast cancer protein and recently has been revealed to belong to a ZIP zinc ion transporter subfamily (Zrt⁻, Irt-like protein) called LZT (LIV-1 subfamily of ZIP zinc ion transporter) (Taylor, K. M. & Nicholson, R. I. The LZT proteins; the LIV-1 subfamily of zinc transporters. Biochim. Biophys. Acta 1611, 16-30 (2003)), and to function as a zinc ion transporter (Taylor, K. M., Morgan, H. E., Johnson, A., Hadley, L. J. & Nicholson, R. I. Structure-function analysis of LIV-1, the breast cancer-associated protein that belongs to a new subfamily of zinc transporters. Biochem. J. 375, 51-9 (2003)). In addition, CDF (cation diffusion facilitator) and the like are included. For example, the expression of the zinc ion transporter LIV1 is regulated by STAT3 (Yamashita, S., Miyagi, C., Fukada, T., Kagara, N., Che, Y.-S. & Hirano, T. Zinc transporter LIVI controls epithelial-mesenchymal transition in zebrafish gastrula organizer. Nature 429, 298-302 (2004)).

The “substance regulating the expression and/or function of a zinc ion transporter” may be a known compound or a novel compound to be developed in the future. It may be a low-molecular-weight compound or a high-molecular-weight compound. In this context, the low-molecular-weight compound is a compound having a molecular weight of less than approximately 3000, which includes, for example, an organic compound and a derivative thereof and an inorganic compound usually available as a pharmaceutical agent, and refers to a compound and a derivative thereof produced by making full use of organic synthesis or the like, a naturally occurring compound and a derivative thereof, small nucleic acid molecules such as promoter, a variety of metals and the like, and desirably an organic compound and a derivative thereof and a nucleic acid molecule available as a pharmaceutical agent. The high-molecular-weight compound is a compound having a molecular weight of not lower than approximately 3000, which includes, for example, protein, polynucleic acid, polysaccharide and combinations thereof, and is desirably a protein. These low-molecular-weight or high-molecular-weight compounds, if they are known, are commercially available or can be obtained by way of steps such as collection, production and purification according to the respective reported documents. These compounds may be naturally occurring or prepared by genetic engineering, or can also be obtained by semi-synthesis and the like.

Since the zinc ion transporter LIV1 enhances the activity (particularly, repressor activity) of the zinc ion-requiring protein Snail (Yamashita, S., Miyagi, C., Fukada, T., Kagara, N., Che, Y.-S. & Hirano, T. Zinc transporter LIVI controls epithelial-mesenchymal transition in zebrafish gastrula organizer. Nature 429, 298-302 (2004)), an example of the “substance regulating the expression and/or function of a zinc ion-requiring protein” in the present invention includes the following:

(1) DNA according to any of the following a) to d):

a) DNA encoding a protein (LIV1 protein) having an amino acid sequence described in SEQ ID NO: 2, 4, or 6;

b) DNA (LIV1 gene) consisting of a sequence described in SEQ ID NO: 1, 3, or 5;

c) DNA encoding a protein (protein analogous to LIV1) having an amino acid sequence described in SEQ ID NO: 2, 4, or 6 with substitution, deletion, insertion and/or addition of one or more amino acid sequences therein;

d) DNA hybridizing to a sequence described in SEQ ID NO: 1, 3, or 5 under stringent condition;

(2) a vector in which DNA according to any of the following a) to d) is inserted:

a) DNA encoding a protein having an amino acid sequence described in SEQ ID NO: 2, 4, or 6;

b) DNA consisting of a sequence described in SEQ ID NO: 1, 3, or 5;

c) DNA encoding a protein having an amino acid sequence described in SEQ ID NO: 2, 4, or 6 with substitution, deletion, insertion and/or addition of one or more amino acid sequences therein;

d) DNA hybridizing to a sequence described in SEQ ID NO: 1, 3, or 5 under stringent condition;

(3) a protein encoded by DNA according to any of the following a) to d):

a) DNA encoding a protein having an amino acid sequence described in SEQ ID NO: 2, 4, or 6;

b) DNA consisting of a sequence described in SEQ ID NO: 1, 3, or 5;

c) DNA encoding a protein having an amino acid sequence described in SEQ ID NO: 2, 4, or 6 with substitution, deletion, insertion and/or addition of one or more amino acid sequences therein;

d) DNA hybridizing to a sequence described in SEQ ID NO: 1, 3, or 5 under stringent condition;

(4) An antisense oligonucleotide whose target sequence is DNA consisting of a sequence described in SEQ ID NO: 1, 3, or 5;

(5) double-stranded RNA having a sequence identical or similar to a portion of DNA consisting of a sequence described in SEQ ID NO: 1, 3, or 5;

(6) an antibody against a protein having an amino acid sequence described in SEQ ID NO: 2, 4, or 6;

(7) a substance (including natural and non-natural products) that associates with a protein having an amino acid sequence described in SEQ ID NO: 2, 4, or 6 and regulates a function of the protein.

The substances (1) to (3) positively regulate the expression and/or function of the zinc ion-requiring protein Snail (enhancing the expression and/or activating the function), and the substances (4) and (5) negatively regulate the expression and/or function of Snail (reducing the expression and/or suppressing the function).

The LIV1 protein can be prepared by a variety of methods well known to those skilled in the art. For example, the protein can be produced by a transformant and obtained by purification thereof, wherein the transformant bears a vector in which DNA (LIV1 gene) consisting of a base sequence described in SEQ ID NO: 1, 3, or 5 is inserted. The vector to be used can be appropriately selected depending on translation systems used for the protein production. Moreover, LIV1 is known to be expressed in hormonal tissues such as the breast, prostate, pituitary gland and brain (Taylor, K. M. & Nicholson, R. I. The LZT proteins; the LIV-1 subfamily of zinc transporters. Biochim. Biophys. Acta 1611, 16-30 (2003)). The LIV1 protein can be purified from LIV1-expressing cell extracts by preparing anti-LIV1 protein antibodies by a well known method and producing an affinity column with the antibodies.

A protein analogous to LIV1 is a protein capable of regulating the activity of a zinc ion-requiring protein (e.g., Snail) and can include, for example, a protein consisting of an amino acid sequence described in SEQ ID NO: 2, 4, or 6 with substitution, deletion, insertion and/or addition of one or more amino acid sequences therein, and a protein encoded by DNA hybridizing to DNA consisting of a base sequence described in SEQ ID NO: 1, 3, or 5 under stringent condition.

The protein analogous to LIV1 can be prepared for example, by a method involving performing hybridization utilizing a base sequence encoding LIV1, producing a transformant with the obtained DNA with high homology and allowing the transformant to produce the desired protein. An example of a method for obtaining the DNA with high homology can include a method involving performing hybridization under stringent hybridization condition using a portion of the base sequence described in SEQ ID NO: 1, 3, or 5 as a probe to obtain such a DNA from cells or the like of human or non-human vertebrate.

Those skilled in the art can appropriately select the above stringent hybridization condition. By way of example, prehybridization is performed overnight at, 42° C. in a hybridization solution containing 25% formamide or 50% formamide for more stringent condition, 4×SSC, 50 mM Hepes pH 7.0, 10×Denhardt's solution, and 20 μg/mL denatured salmon sperm DNA, and then hybridization is performed by adding a labeled probe and incubating overnight at 42° C. Subsequent washing can be performed under condition of a washing liquid and temperature of approximately “1×SSC, 0.1% SDS, at 37° C.”, approximately “0.5×SSC, 0.1% SDS, at 42° C.” for more stringent condition, and approximately “0.2×SSC, 0.1% SDS, at 65° C.” for even more stringent condition. Thus, it can be expected that the more stringent the washing condition of the hybridization is, the higher the homology of the isolated DNA to the probe sequence is. However, the combinations of the SSC, SDS and temperature conditions are described for purposes of only exemplification, and those skilled in the art can appropriately combine the above-described or other determinants of the stringency of hybridization (e.g., probe concentration, probe length, hybridization reaction period and the like), thereby achieving a stringency similar to those described above.

A polypeptide encoded by the DNA isolated using such a hybridization technique generally has high homology to LIV1 in the amino acid sequence. The high homology refers to at least 40% or higher, preferably 60% or higher, more preferably 80% or higher, even more preferably 90% or higher, even still more preferably 95% or higher, particularly preferably 97% or higher (e.g., 98 to 99%) of sequence homology. An amino acid sequence identity can be determined by, for example, algorithm BLAST (Karlin and Altschul, Proc. Natl. Acad. Sci. USA 87: 2264-2268, 1990; and Proc. Natl. Acad. Sci. USA 90: 5873-5877, 1993). Based on this algorithm, a program called BLASTX has been developed (Altschul et al., J. Mol. Biol. 215: 403-410, 1990). When amino acid sequences are analyzed with the use of BLASTX, parameters are set to, for example, score=50 and word length=3. When BLAST and Gapped BLAST programs are used, the respective default parameters of the programs are used. The specific ways of these analysis methods are known (http://www.ncbi.nlm.nih.gov.)

The protein analogous to LIV1 may also be prepared by another known means. For example, LIV1 DNA is artificially modified by deletion mutant production using exonuclease and site-directed mutagenesis such as cassette mutagenesis, and the modified LIV1 DNA can be used to prepare the desired protein.

Another preferable example of the substance available as the “substance regulating the expression and/or function of a zinc ion-requiring protein” can include DNA consisting of the sequence described in SEQ ID NO: 1, 3, or 5. The above described DNA encodes LIV1 protein, and when introduced into cells, serves as a substance capable of indirectly regulating the activity of a zinc ion-requiring protein such as Snail, through LIV1 protein expression.

The above described DNA can also be prepared from a cDNA of a LIV1-expressing cell by hybridization techniques well known to those skilled in the art using a portion of the sequence described in SEQ ID NO: 1, 3, or 5 as a probe. The DNA can also be obtained from mRNA by RT-PCR using a portion of the sequence described in SEQ ID NO: 1, 3, or 5 as a primer. Alternatively, the DNA may also be synthesized artificially with a commercially available DNA synthesizer.

Furthermore, DNA similar to the above DNA is also an example of the “substance regulating the expression and/or function of a zinc ion-requiring protein”. The similar DNA can include DNA hybridizing to the sequence described in SEQ ID NO: 1, 3, or 5 under stringent condition and encoding the substance regulating the expression and/or function of a zinc ion-requiring protein, for example, a protein capable of regulating the activity of Snail. The DNA can be prepared as previously described above.

When each DNA described above is used, the DNA can be inserted into an appropriate vector and used. The DNA inserted into the vector is also one of the aspects of the present invention. As a vector to be used, a suitable vector can be selected depending on the purpose. Particularly, the vector can include mammal-derived vectors (e.g., pcDNA3 (manufactured by Invitrogen), pEGF-BOS (Nucleic Acids. Res., 18(17), p. 5322, 1990), pEF, pCDM8, PCXN), insect cell-derived vectors (e.g., “Bac-to-BAC baculovirus expression system” (manufactured by Invitrogen), pBacPAK8), plant-derived expression vectors (e.g., pMH1, pMH2), animal virus-derived vectors (e.g., pHSV, pMV, pAdexLcw), retrovirus-derived vectors (e.g., pZIPneo), yeast-derived vectors (e.g., “Pichia Expression Kit” (manufactured by Invitrogen), pNV11, SP-Q01), Bacillus subtilis-derived vectors (e.g., pPL608, pKTH50), Escherichia coli vectors (M13-type vectors, pUC-type vectors, pBR322, pBluescript, pCR-Script) and the like. In the present invention, it is preferred to use a vector capable of being expressed in a mammal cell and to use an expression vector. The vector can be introduced into a cell by a method selected from, for example, calcium phosphate method (Virology, Vol. 52, p. 456, 1973), DEAE dextran method, a method using cationic liposome DOTAP (manufactured by Roche Diagnostics), electroporation method (Nucleic Acids Res., Vol. 15, p. 1311, 1987), lipofection method (J. Clin. Biochem. Nutr., Vol. 7, p. 175, 1989), introduction method by viral infection (e.g., pMX, pMSCV; Sci. Am., p. 34, 1994), a particle gun and the like.

One example of the “substance regulating the expression and/or function of a zinc ion-requiring protein”, for example, an agent suppressing the activity of Snail can include an antisense oligonucleotide targeting DNA or mRNA encoding LIV1. It is believed that the antisense oligonucleotide against the DNA encoding LIV1 prevents the expression of an endogenous LIV1 gene and negatively control the Snail activity. Such an antisense oligonucleotide can include an antisense oligonucleotide whose target sequence is DNA consisting of a sequence described in SEQ ID NO: 1, 3, or 5 or mRNA sequence generated from the DNA sequence. An example thereof can include an oligonucleotide described in SEQ ID NO: 7 or 8. In addition, any antisense oligonucleotide that can hybridize to any site in the DNA consisting of the above sequence described in SEQ ID NO: 1, 3, or 5, and the like, and can effectively inhibit LIV1 expression is included in the antisense oligonucleotide of the present invention. Such an antisense oligonucleotide is not necessarily completely complementary to the DNA consisting of the above sequence described in SEQ ID NO: 1, 3, or 5, or the corresponding mRNA.

The above described antisense oligonucleotide can be inserted into a suitable vector depending on the purposes and used. For example, for the purpose of applying it to a gene therapy, the vector may be selected appropriately from viral vectors such as retroviral, adenoviral and vaccinia viral vectors, and non-viral vectors such as cationic liposomes and ligand-DNA complexes, and the like. It can also be administered as a naked plasmid DNA (naked pDNA) together with a large volume of aqueous solutions without the use of carriers.

Another example of the “substance regulating the expression and/or function of a zinc ion-requiring protein”, particularly the agent suppressing the activity of Snail can include double-stranded RNA having a sequence identical or similar to a portion of DNA encoding LIV1. A double-stranded RNA having a sequence identical or similar to a target gene sequence is capable of inducing RNA interference (RNAi) that inhibits the expression of the target gene. The RNAi refers to a phenomenon in which when double-stranded RNA (dsRNA) is introduced into a cell, an intracellular mRNA corresponding to the RNA sequence is specifically degraded and as a result is not expressed as a protein. The double-stranded RNA may be entirely composed of a region forming double strand or may have a partial region forming single strand (e.g., at both ends or either end) and the like. Thus, the double-stranded RNA of the present invention may also contain a region that is not double-stranded. An oligo RNA used in RNAi is often 10- to 100-bp RNA, and usually 19- to 23-bp RNA. The RNAi can be performed according to the descriptions of Nature, Vol. 391, p. 806, 1998, Proc. Natl. Acad. Sci. USA Vol. 95, p. 15502, 1998, Nature, Vol. 395, p. 854, 1998, Proc. Natl. Acad. Sci. USA Vol. 96, p. 5049, 1999, Cell, Vol. 95, p. 1017, 1998, Proc. Natl. Acad. Sci. USA

Vol. 96, p. 1451, 1999, Proc. Natl. Acad. Sci. USA Vol. 95, p. 13959, 1998, Nature Cell Biol., Vol. 2, p. 70, 2000, and the like.

The “substance regulating the expression and/or function of a zinc ion transporter” is, for example, when the substance is LIV1, exemplified by those similar to the substances described above for the “substance regulating the expression and/or function of a zinc ion-requiring protein”.

When the “zinc ion transporter” is other than LIV1, for example, hZnt-1, hZnt-2, hZnt-5, hZnt-6, hZnt-7, hZIP1, hZIP2, hZIP3, BAA92100, BAC04504, hLiv-1, hKE4, KIAA1265, KIAA0062, or hZip4, a variety of “substances regulating the expression and/or function of a zinc ion transporter” or “substances regulating the expression and/or function of a zinc ion-requiring protein” can be prepared in the same way as for LIV1, based on the known amino acid sequence or base sequence of the zinc ion transporter.

Information about the respective amino acid sequences and base sequences can be obtained from a variety of databases browsable in NCBI and the like.

The function controlling agent of the present invention for an antigen-presenting cell and the prophylactic and/or therapeutic drug of the present invention for a disease mediated by the immune system comprise, if desired, pharmaceutically acceptable excipients and additives, in addition to the above described series of active ingredients (zinc ion, zinc ionophore, zinc ion chelator, substance regulating the expression and/or function of a zinc ion-requiring protein, and substance regulating the expression and/or function of a zinc ion transporter). The pharmaceutically acceptable excipients and additives include carriers, binders, flavors, buffers, thickeners, coloring agents, stabilizers, emulsifiers, dispersants, suspending agents, preservatives and the like. Moreover, different types of “substances controlling the zinc ion concentration” can be used in combination as active ingredients.

The pharmaceutically acceptable carriers include, for example, magnesium carbonate, magnesium stearate, talc, sugar, lactose, pectin, dextrin, starch, gelatin, tragacanth, methylcellulose, sodium carboxymethylcellulose, low-melting wax, cocoa butter and the like.

Furthermore, tablets can be made, if necessary, into tablets coated with usual coatings, for example sugar-coated tablets, enteric-coated tablets, film-coated tablets, or two-layer tablets or multi-layer tablets. Powders can be prepared with pharmaceutically acceptable bases for powders. The bases include talc, lactose, starch and the like. Drops can be prepared together with aqueous or non-aqueous bases and one or more pharmaceutically acceptable dispersants, suspending agents, solubilizers, and the like. Capsules can be prepared by filling with a compound serving as an active ingredient together with a pharmaceutically acceptable carrier. The compound can be mixed with or without a pharmaceutically acceptable excipient and filled into capsules. Cachets can also be prepared in a similar way. When the present invention is prepared as a suppository, the suppository is prepared together with bases such as vegetable oil (e.g., castor oil, olive oil, peanut oil), mineral oil (e.g., petrolatum, white petrolatum), waxes, and partially or fully synthesized glycerin fatty acid ester, by an approach generally used.

Liquid formulations for injection include solutions, suspensions, emulsions and the like. Examples thereof include aqueous solutions, water-propylene glycol solutions and the like. The liquid formulations can also be produced in the form of a solution of polyethylene glycol and/or propylene glycol, which may contain water.

Liquid formulations suitable for oral administration can be produced by adding the compound serving as an active ingredient and, if necessary, a coloring agent, flavor, stabilizer, sweetening agent, solubilizer, thickener, or the like, to water. Alternatively, the liquid formulations suitable for oral administration can be produced by adding the compound and a dispersant to water to give a viscous liquid. Examples of the thickener include pharmaceutically acceptable natural or synthetic gum, resin, methylcellulose, sodium carboxymethylcellulose, known suspending agents and the like.

Agents for local administration include the liquid formation described above, and cream, aerosol, spray, powder, lotion, ointment and the like. The above described agents for local administration can be produced by mixing a compound serving as an active ingredient with pharmaceutically acceptable diluents and carriers. The ointment and cream can be prepared, for example, by adding thickeners and/or gelling agents to an aqueous or oily base. Examples of the base include water, liquid paraffin, vegetable oil and the like. Examples of the thickener include soft paraffin, aluminum stearate, cetostearyl alcohol, propylene glycol, polyethylene glycol, lanolin, hydrogenated lanolin, beeswax and the like. To the agent for local administration can be added, if necessary, a preservative such as methyl hydroxybenzoate, propyl hydroxybenzoate, chlorocresol, benzalkonium chloride and the like, or a bacterial growth inhibitor. The lotion can be prepared by adding one or more pharmaceutically acceptable stabilizers, suspending agents, emulsifiers, dispersants, thickeners, coloring agents, flavors, and the like, to an aqueous or oily base.

The thus-obtained function controlling agent of the present invention for an antigen-presenting cell and the prophylactic and/or therapeutic drug of the present invention for a disease mediated by the immune system are orally or parenterally administered.

When administered orally, they can be administered in a dosage form commonly used in the art. When administered parenterally, they can be administered in a dosage form such as agent for local administration (e.g., transdermal agent), agent for rectal administration, injectable, transnasal agent and the like.

Examples of the oral agent or agent for rectal administration include capsule, tablet, pill, powder, drop, cachet, suppository, liquid formulation and the like. Examples of the injectable include aseptic solutions or suspensions and the like. Examples of the agent for local administration include cream, ointment, lotion, transdermal agent (general patch agent or matrix agent) and the like.

The dosage and the number of administration may vary depending on the type of the substance capable of controlling the zinc ion concentration used, the condition, age and body weight of a patient, a dosage form and the like, and can be determined appropriately.

All of the “zinc ion”, “zinc ion chelator”, “substance regulating the expression and/or function of a zinc ion-requiring protein”, and “substance regulating the expression and/or function of a zinc ion transporter” are capable of controlling the function of an antigen-presenting cell, specifically the expression of a molecule associated with antigen presentation, activation of an antigen-presenting cell by LPS and the like, cytokine production, antigen uptake, migration capability and the like in humans and other mammals such as cattle, horses, dogs, mice, and rats, and hence make it possible to prevent/treat a disease involved in the function of an antigen-presenting cell, specifically a disease mediated by the immune system. As the disease mediated by the immune system, autoimmune disease, allergic disease, cancer, graft rejection in organ transplant, immune response reductions due to infection or tumorigenesis, abnormal immune responses during vaccination (for example, adverse reactions) and the like can be mentioned. For example, the present invention is expected to be useful for treatment of SIRS (systemic inflammatory reaction syndrome), systemic lupus erythematosus, mixed connective tissue disease, rheumatoid arthritis, Sjogren's syndrome, rheumatic fever, Goodpasture syndrome, Basedow disease, Hashimoto disease, Addison disease, autoimmune hemolytic anemia, idiopathic thrombocytopenic purpura, myasthenia gravis, ulcerative colitis, Crohn disease, sympathetic ophthalmia, multiple sclerosis, psoriasis, anaphylactic shock, allergic rhinitis, allergic conjunctivitis, bronchial asthma, urticaria, atopic dermatitis and the like, for pre-transplant treatment or post-transplant administration for suppressing graft rejection in organ transplant, and for control of immune responses to cancer, and the like. As the cancer, cancers such as breast cancer, prostatic cancer, pancreatic cancer, gastric cancer, lung cancer, colorectal cancer (colic cancer, rectal cancer, anal cancer), esophageal cancer, duodenal cancer, cancer of head and neck (cancer of tongue, cancer of pharynx), brain tumor, schwannoma, non-small-cell lung cancer, small-cell lung cancer, liver cancer, kidney cancer, cancer of bile duct, uterine cancer (uterine body cancer, uterine cervical cancer), ovarian cancer, urinary bladder cancer, skin cancer, hemangioma, malignant lymphoma, malignant melanoma, thyroid cancer, bone tumor, hemangioma, angiofibroma, retinal sarcoma, penile cancer, solid cancers in children, Kaposi's sarcoma, AIDS-associated Kaposi's sarcoma, maxillary sinus tumor, fibrous histiocytoma, smooth muscle sarcoma, and rhabdomyosarcoma, and malignant tumors such as leukemia and the like can be mentioned.

By introducing a zinc ion into an antigen-presenting cell by means of a drug such as a zinc ionophore, it is possible to suppress the activation of an antigen-presenting cell by LPS and the like, and to suppress cytokine production, and even to control the balance between Th1 and Th2, and CTL and B cell activation. That is, the function controlling agent of the present invention for an antigen-presenting cell and the prophylactic and/or therapeutic drug of the present invention for a disease mediated by the immune system can be administered even to patients suffering from various diseases for which a therapeutic effect is observed when a zinc ion is introduced into an antigen-presenting cell.

Furthermore, based on the finding obtained by the present inventors that the function of an antigen-presenting cell can be controlled by controlling the zinc ion concentration, a compound capable of controlling the function of an antigen-presenting cell can be screened for by measuring the intracellular zinc ion concentration. This screening method is performed by, for example, the following steps:

1) a step for dividing antigen-presenting cells into two groups, and treating one group with a test compound (the remaining untreated group is used as a control);

2) a step for measuring the respective intracellular zinc ion concentrations for the treated cells and the untreated control cells; and

3) a step for selecting a compound that caused a significant change in zinc ion concentration compared to the zinc ion concentration in the control cell, and judging the compound as being capable of controlling the function of an antigen-presenting cell.

As the antigen-presenting cell, one of those described above is used. The antigen-presenting cell is preferably a dendritic cell. The cells are randomly divided into two groups, and one of which is treated with a test compound. The other is untreated and used as a control cell. Furthermore, a compound known to influence the zinc ion concentration, for example, the zinc ion chelator TPEN and the like, may be used as a positive control compound. The test compound may be a known compound or a novel compound to be developed in the future. It may be a low-molecular-weight compound or a high-molecular-weight compound. In this context, the low-molecular-weight compound is a compound having a molecular weight of less than approximately 3000, which includes, for example, an organic compound and a derivative thereof and an inorganic compound usually available as a pharmaceutical agent, and refers to a compound and a derivative thereof produced by making full use of organic synthesis or the like, a naturally occurring compound and a derivative thereof, small nucleic acid molecules such as promoter, a variety of metals and the like, and desirably an organic compound and a derivative thereof and a nucleic acid molecule available as a pharmaceutical agent. The high-molecular-weight compound is a compound having a molecular weight of not lower than approximately 3000, which includes, for example, protein, polynucleic acid, polysaccharide and combinations thereof, and is desirably a protein. These low-molecular-weight or high-molecular-weight compounds, if they are known, are commercially available or can be obtained by way of steps such as collection, production and purification according to the respective reported documents. These compounds may be naturally occurring or prepared by genetic engineering, or can also be obtained by semi-synthesis and the like.

The time period of the treatment of cells with the test compound is appropriately set depending on the types and concentrations of cells and test compounds to be used. When the positive control compound such as TPEN is used, the treatment can be performed using, as a guide, a change in the zinc ion concentration when treated with the control compound. The concentration of the test compound for treating cells is also appropriately set depending on the types of the cells and test compounds to be used, as well as the treatment period.

The measurement of the intracellular zinc ion concentration can be performed utilizing a method routinely practiced in the art or a method pursuant thereto. The concentration can be measured, for example, by direct measurement by atomic absorption method (flame method) or with a spectrophotofluorometer using a specific probe (e.g., fluorescent reagent).

### EXAMPLES

Hereinafter, although the present invention will be described in more detail with reference to Examples, these Examples do not limit the scope of the present invention by any means. All the publications cited throughout the present application are incorporated herein by reference. The reagents, apparatuses and materials used in the present invention are commercially available unless otherwise specified.

### Example 1

Dendritic cells, a type of antigen-presenting cells, were treated with N,N,N′,N′-tetrakis(2-pyridylmethyl)ethylenediamine (TPEN), a zinc ion chelator, and examined for the expression of MHC class I molecule, class II molecule and CD86 molecule on the cell surface.

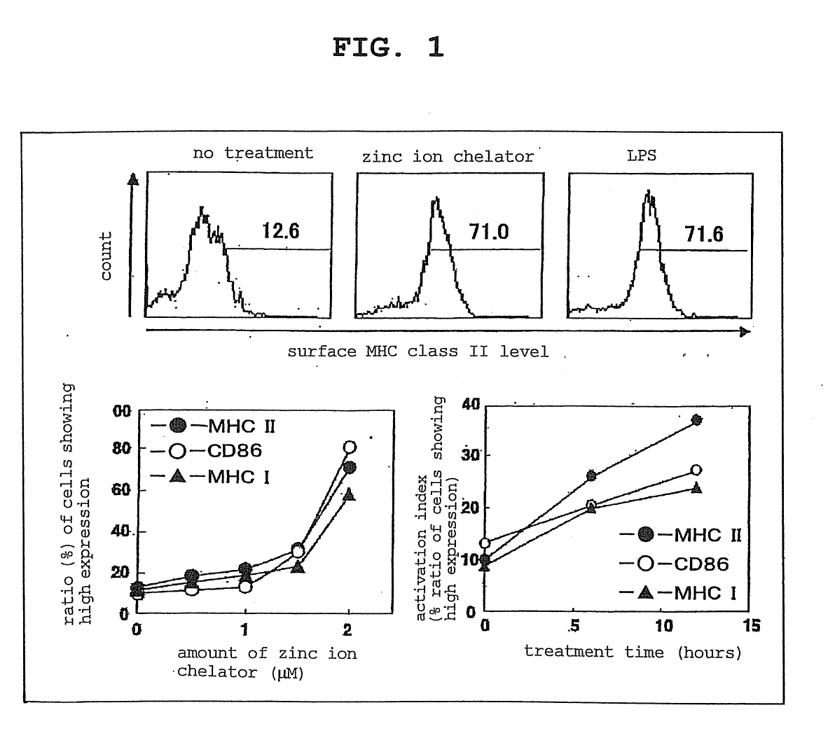

Bone marrow cells were obtained from a C57BL/6 mouse (obtained from Clea Japan) and subjected to cell culture using an RPMI medium (SIGMA, R8758) supplemented with 10% FCS (EQUITECH-BIO Inc., #SFB-30-1388) and granulocyte macrophage colony stimulating factor (GM-CSF) in a 5% CO₂ environment at 37° C. for 6 days to induce the differentiation of bone marrow-derived dendritic cells (BMDC). To this BMDC, TPEN (SIGMA, P4413) was added for 6 hours to 12 hours to obtain concentrations of 0-2 μM in the medium. Also prepared was a group in which lipopolysaccharide (LPS; 100 ng/mL; SIGMA, L2637, E. Coli O55:B5) was added for control of antigen-presenting cell activation. The MHC class I, class II and CD86 molecules that became expressed on the cell surface after 12 hours (or 6 hours) were evaluated by FACS analysis. The results are shown in FIG. 1. Each numerical value in FIG. 1 shows the percentage of cells highly expressing each molecule. When the cells were treated with the zinc ion chelator, the expression of MHC class II molecule on BMDC was enhanced as with LPS. This expression enhancement was observed with drug dose dependency (treatment time of 12 hours) and treatment time dependency (treatment concentration of 1.5 μM).

### Example 2

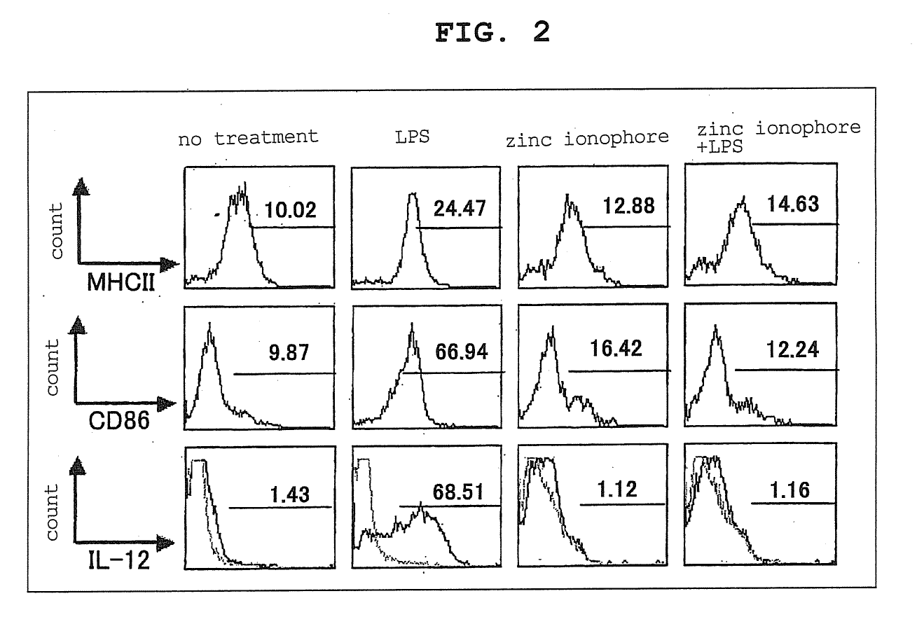

The zinc ionophore pyrithione (5 μM; Molecular probes, p-24193) and Zn²⁺ (ZnSO₄) were added to BMDC prepared in the same manner as Example 1 to obtain a final concentration of 5 μM in the culture broth, and the BMDC was further stimulated with LPS (100 ng/mL). The MHC class II and CD86 molecules that became expressed on the cell surface after 6 hours were evaluated by FACS analysis. The results are shown in FIG. 2. Each numerical value in FIG. 2 shows the percentage of cells highly expressing each molecule. At the same time, IL-12 production by the BMDC was detected by the intracellular staining method using an anti-IL-12p40 antibody (BD.Bioscience Pharmingen, #554479) and an isotype control antibody (BD.Bioscience Pharmingen, #553925) after fixation with 4% para-formaldehyde. Each numerical value in the figure shows the percentage of cells that produced IL-12.

No expression enhancement of MHC II and CD86 molecules on the cell surface or IL-12 cytokine production that accompany LPS-stimulated BMDC activation was observed in the presence of the zinc ionophore.

In the same experiment (in vitro system) as Examples 1 and 2, dendritic cell activation or inactivation was observed by a treatment with 1-10 μM zinc ion or TPEN.

### Example 3

Using a retrovirus vector that expresses mouse Liv1 as the substance that controls the zinc ion concentration, its effect on LPS-stimulated expression enhancement of MHC class II molecule was examined.

A clone comprising the mouse Liv1 gene (mLiv1, gene sequence: SEQ ID NO:5, amino acid sequence: SEQ ID NO:6) supplied by RIKEN (mfj04623) was cleaved with the restriction endonucleases XhoI and NotI, and the resulting mLiv1 gene was recovered and inserted in the pMX-IRES-GFP retrovirus vector prepared and supplied by Professor Toshio Kitamura at the University of Tokyo Institute of Medical Science. This pMX-IRES-GFP-mLiv1 vector was introduced into Phoenix cells supplied by Dr. Garry P. Nolan at Stanford University of USA using lipofectamine (Invitrogen) to express the virus. After 24 hours to 48 hours, the culture supernatant containing this virus was recovered, GM-CSF [a GM-CSF-producing cell line (GM-CSF/CHO) was cultured, and the culture supernatant was added to the medium to obtain a concentration of 0.3%], FCS (10%), and protamine sulfate (SIGMA; 2 μg/mL) were added, and the mixture was used as the virus-infection liquid. BMDC prepared from the bone marrow of a C57BL/6 mouse (Clea Japan) were infected by exchanging the medium with the virus-infection liquid on Day 2 and Day 4. On Day 6, the liquid was exchanged with a virus-free medium, and the BMDC was further stimulated with LPS (SIGMA; 100 ng/mL). Six hours after the stimulation, the cells were recovered, and the expression level of MHC class II molecule on the cell surface was examined by FACS. Here, a group of cells not stimulated with LPS were handled as controls, and GFP-positive cells as cells showing forced expression of mLiv1.

It was found that expression enhancement of MHC class II molecule by LPS stimulation was suppressed in cells showing forced expression of mLiv1.

### Free-Text of Sequence Listing

SEQ ID No: 7: Antisense sequence
SEQ ID No: 8: Antisense sequence

### INDUSTRIAL APPLICABILITY

In an antigen-presenting cell, by controlling the zinc ion concentration, or by regulating the expression or function of a zinc ion-requiring protein and a zinc ion transporter, the expression of a molecule associated with antigen presentation (for example, MHC class I, class II, CD86 molecule and the like) can be enhanced. Furthermore, by introducing a zinc ion into an antigen-presenting cell by means of a drug such as a zinc ionophore, activation of an antigen-presenting cell and cytokine production by lipopolysaccharide (LPS) and the like can be suppressed. Therefore, simply by regulating the intracellular level of zinc essentially present in the body, without using conventional immunopotentiating molecules, immunosuppressants and the like such as microbial constituents and synthetic steroids, it is possible, for example, to enhance or suppress the immune system via activation of an antigen-presenting cell such as a dendritic cell.

This application is based on a patent application No. 2005-023200 filed in Japan on Jan. 31, 2005, the contents of which are incorporated in full herein by this reference.

1. An agent for controlling the function of an antigen-presenting cell, comprising a substance capable of controlling the intracellular zinc ion concentration as the active ingredient.

2. The agent of claim 1, wherein the substance capable of controlling the zinc ion concentration is a zinc ion.

3. The agent of claim 2, wherein the zinc ion is introduced into a cell through a zinc ionophore.

4. The agent of claim 1, wherein the substance capable of controlling the zinc ion concentration is a zinc ion chelator.

5. The agent of claim 4, wherein the zinc ion chelator is at least one selected from the group consisting of 2,3-dimercapto-1-propanesulfonic acid (DMPS), N,N,N′,N′-tetrakis(2-pyridylmethyl)ethylenediamine (TPEN), ethylenediamine (EDTA) and N-(6-methoxy-8-quinolyl)-p-toluenesulfonamide (TSQ).

6. The agent of claim 1, wherein the substance capable of controlling the zinc ion concentration is a substance regulating the expression and/or function of a zinc ion-requiring protein.

7. The agent of claim 6, wherein the zinc ion-requiring protein is at least one selected from the group consisting of Raf-1, PKCα, GEF-H1, HDAC, SQSTM1, ubiquitin-protein ligase E3, ubiquitin-conjugating enzyme E2, metallothionein, phosphatase, Snail, TRAF6, Paxillin, Zyxin and Jade-1.

8. The agent of claim 1, wherein the substance capable of controlling the zinc ion concentration is a substance regulating the expression and/or function of a zinc ion transporter.

9. The agent of claim 8, wherein the zinc ion transporter is at least one selected from the group consisting of human ZIPs including LIV family and human CDFs.

10. The agent of claim 1, wherein the antigen-presenting cell is at least one selected from the group consisting of a dendritic cell, a macrophage, a Langerhans' cell, a B cell, a thymic epithelial cell, a synovial cell, a vascular endothelial cell and a keratinocyte.

11. The agent of claim 10, wherein the antigen-presenting cell is a dendritic cell.

12. A prophylactic and/or therapeutic drug for a disease mediated by the immune system, comprising the function controlling agent of claim 1.

13. The prophylactic and/or therapeutic drug of claim 12, wherein the disease mediated by the immune system is at least one selected from the group consisting of autoimmune disease, allergic disease, graft rejection in organ transplant, an immune response reduction due to infection or tumorigenesis, and an abnormal immune response during vaccination.

14. A prophylactic and/or therapeutic drug for a disease mediated by the immune system, comprising a substance capable of controlling the intracellular zinc ion concentration as the active ingredient.

15. The prophylactic and/or therapeutic drug of claim 14, wherein the substance capable of controlling the zinc ion concentration is a zinc ion.

16. The prophylactic and/or therapeutic drug of claim 15, wherein the zinc ion is introduced into a cell through a zinc ionophore.

17-25. (canceled)

26. A method of controlling the function of an antigen-presenting cell, comprising controlling the intracellular zinc ion concentration in vitro.

27. The method of claim 26, wherein the zinc ion concentration is controlled by introducing a zinc ion into a cell.

28-38. (canceled)

39. A use of a substance capable of controlling the intracellular zinc ion concentration, for producing a function controlling agent for an antigen-presenting cell.

40. A use of the function controlling agent of claim 1, for producing a prophylactic and/or therapeutic drug for a disease mediated by the immune system.

41. (canceled)
